(19) United States
(12) Plant Patent Application Publication
(10) Pub. No.: US 2010/0313318 P1
(43) Pub. Date: Dec. 9, 2010

(54) ECHEVERIA PLANT NAMED 'CRIMSON TIDE'

(50) ;

(76) Inventor: Renee O'Connell, Escondido, CA (US)

Correspondence Address: HortisUSA, PO Box 3109, Camarillo, CA, 93011, US

(21) Appl. No.: 12/455,707

(22) Filed: Jun. 6, 2009

(51) Int. Cl. A01H 5/00

(52) U.S. Cl. PLT/263.1

(57) ABSTRACT

A new and distinct Echeveria cultivar named ‘Crimson Tide’ is disclosed, characterized unique vividly colored leaf margins which are unusually undulating and thick. Leaf margins are also distinctively anfractuous. The new variety is an Echeveria, typically produced as a garden or container plant.

### LATIN NAME OF THE GENUS AND SPECIES

Echeveria hybrid

### VARIETY DENOMINATION

‘Crimson Tide’

### BACKGROUND OF THE INVENTION

The new cultivar is a product of a planned breeding program. The new variety originated from a cross pollination of the unpatented, proprietary seed parent, Echeveria hybrid referred to as ‘#26’ with the pollen parent an unpatented proprietary Echeveria hybrid referred to as ‘Big Red.’ The crossing was made during October of 2005 in Vista, Calif., at a commercial greenhouse. ‘Crimson Tide’ was discovered by the inventor, Renee O'Connell, in February 2007, in Vista, Calif. at a commercial greenhouse.

Asexual reproduction of the new cultivar ‘Crimson Tide’ was first performed in Vista, Calif., at a commercial greenhouse by vegetative leaf cuttings in February 2007. ‘Crimson Tide’ has since produced several generations and has shown that the unique features of this cultivar are stable and reproduced true to type.

### SUMMARY OF THE INVENTION

The cultivar ‘Crimson Tide’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘Crimson Tide.’ These characteristics in combination distinguish ‘Crimson Tide’ as a new and distinct Echeveria cultivar:
1. Unique vividly colored leaf margins.
2. Unusual undulating, thick leaf margins.
3. Distinctive anfractuous leaf margins.

### PARENTAL COMPARISON

Plants of the new cultivar ‘Crimson Tide’ are similar to the seed parent ‘#26’ in most horticultural characteristics. However, ‘Crimson Tide’ exhibits unusual brightly colored, anfractuous leaf margins not seen on the seed parent. Additionally, ‘Crimson Tide’ grows faster, and shows an overall better resistance to normal diseases and pests of Echeveria than the seed parent.

Plants of the new cultivar ‘Crimson Tide’ are similar to the pollen parent ‘Big Red’ in most horticultural characteristics. However, ‘Crimson Tide’ exhibits more undulating, brighter colored leaf margins than the pollen parent. Additionally, ‘Crimson Tide’ has an overall shorter, more compact plant morphology than ‘Big Red.’

### COMMERCIAL COMPARISON

‘Crimson Tide’ can be compared to the unpatented commercial variety Echeveria ‘Strawberry Hearts.’ Plants of Echeveria ‘Strawberry Hearts’ are similar to plants of ‘Crimson Tide’ in most horticultural characteristics. However, ‘Crimson Tide’ does not grow as tall as ‘Strawberry Hearts’ and produces a more irregular rosette plant form. Additionally, plants of ‘Crimson Tide’ produce a more anfractuous leaf margin than ‘Strawberry Hearts.’

‘Crimson Tide’ can also be compared to the unpatented commercial variety Echeveria ‘Arlie Wright.’ Plants of Echeveria ‘Arlie Wright’ are similar to plants of ‘Crimson Tide’ in most horticultural characteristics. However, ‘Crimson Tide’ is a shorter plant, which exhibits more resistance to the disease powdery mildew than ‘Arlie Wright.’ Additionally, the foliage of ‘Crimson Tide’ has a more intense margin color, and a more undulating, anfractuous form.

### BRIEF DESCRIPTION OF THE PHOTOGRAPH

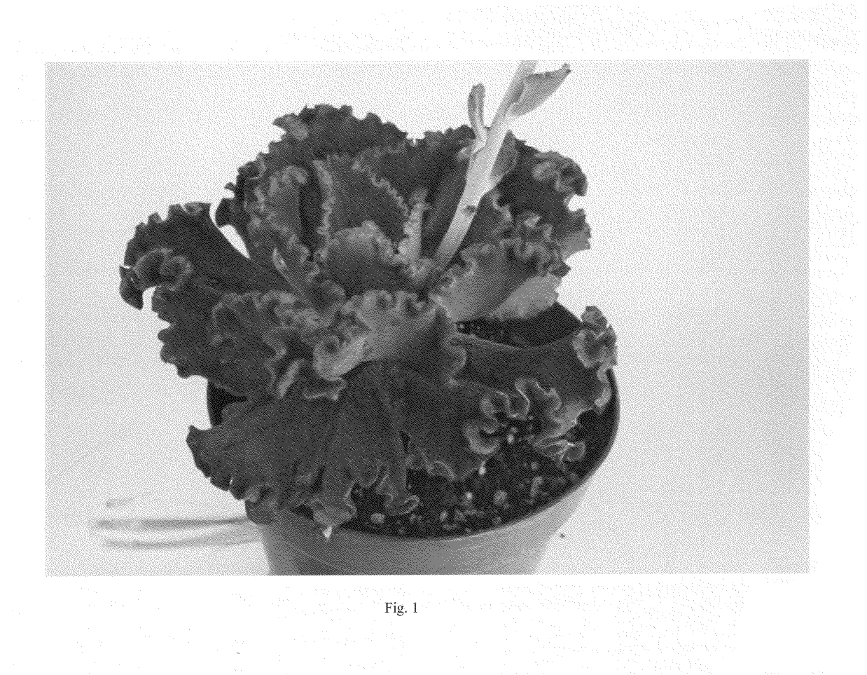

The accompanying photograph in FIG. 1 illustrates in full color a typical plant of ‘Crimson Tide’ grown outdoors in Vista, Calif. This plant is approximately 9 months old, shown in a 6 inch pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to the Pantone Process Color System Guide, except where general terms of ordinary dictionary significance are used. The following observations and measurements describe ‘Crimson Tide’ plants in a commercial greenhouse in Vista, Calif. Temperatures ranged from −1° C. to 29° C. night and day. No artificial light, photoperiodic treatments or chemical treatments were given to the plants. Natural light conditions were approximately 2500 to 4000 fc of light. Measurements and numerical values represent averages of typical plant types.
Botanical classification: Echeveria hybrid ‘Crimson Tide.’
Age of the plant described: Approximately 5 months.
Propagation:
Time to initiate roots: About 25 days at approximately 21° C.
Root description: Fibrous.
Propagation method: Vegetative divisions, leaf cuttings.
Plant:
Growth habit: Rosulate plant, sessile, rarely branching.
Container size: 6 inch.
Height: Approximately 15 cm to top of highest leaf.
Plant spread: Approximately 19 cm.
Growth rate: Slow-moderate
Branching characteristics: Rarely.
Foliage:
Leaf:
   Arrangement.—rosulate; sessile.
   Average length.—Approximately 11 cm.
   Widest width.—Approximately 8.75 cm.
   Width at base.—Approximately 2 cm.
   Shape of blade.—Spatulate.
   Apex.—Rounded, crenulate.
   Base.—Rounded.
   Margin.—Crenulate, anfractuous.
   Texture of top surface.—Glabrous, pruniose.
   Texture of bottom surface.—Glabrous; pruinose.
   Quantity of leaves per plant.—Approximately 20.
   Color.—Young foliage upper side: Near S 137-5 Pantone Young foliage under side: Near S 307-9 Pantone Young foliage margin: Near S 107-8 to S 107-3 Pantone Mature foliage upper side: Near S 137-6 Pantone. Margin near Near S 107-1 Pantone. Mature foliage under side: Near S 137-6 Pantone. Margin near Near S 107-1 Pantone. Mature foliage, under side; if pruinose covering is removed: Near S 291-8 Pantone Mature foliage margin: Near S 107-1 Pantone.
   Venation.—Type: Parallel. Venation color upper side: Indistinguishable from overall foliage color. Venation color under side: Indistinguishable from overall foliage color.
Flower:
Natural flowering season: Late winter, late autumn.
Flowering: Plants were not flowering at the time the botanical description was made, as all plants of the new variety had flowers removed for propagation purposes.
Other characteristics:
Fruits and seeds: Not observed to date.
Temperature tolerance: Tolerates temperatures from approximately −2 C to 32 C.
Disease/pest resistance: Neither resistance nor susceptibility to normal diseases and pests of Echeveria has been observed.
Drought tolerance: Tolerates at least 2 weeks of high temperatures without supplemental water, showing no serious damage to plant.

What is claimed is:

1. A new and distinct cultivar of Echeveria plant named ‘Crimson Tide’ as herein illustrated and described.
